SEQUENCE LISTING (4 sequences)

SEQ ID NO: 1 - DNA, 1366 bp, Strawberry
cggcacgagcatctatctatctatccatctctgaatctgaacgttgttctcttaaaccaaggtatttagccagagacatatatcccacattcgatccattcatttcaattgtaaactagctcaatggcaaaggttccttcagtaaccctcagctcctgcggtgatgacatccagaccatgcctgtaatcggcatgggaacttcatcgtaccctcgggccgaccctgaaaccgccaaggctgctattctcgaagcaattagagctggttaccgacatttcgacaccgccgctgcttacggctcggagaaagatctcggtgaagccatagccgaggctctccgtctccaactcatcaagtctagggacgagctcttcatcacaaccaaactttgggccagtttcgccgagaaagaccttgtgctgccctccatcaaagccagtttaagcaatcttcaagtagaatacattgacatgtacatcatacactggccattcaaattgggaaaagaggtgagaaccatgcctgttgagagagatctggtgcagccccttgatatcaaatctgtttgggaagccatggaagagtgcaagaaacttgggcttgctagaggtattggtgtcagtaacttcactagcagcatgcttgaggagcttctttccttcgccgaaatccctccggtcgtaaaccaattggagatgaacccagcttggcagctgaagaaattgagggacttctgcaaggcaaagggaattcatgtcacggcttactctccgcttggagcagctaggactaaatggggtgacgatagggttttgggatcagatatcatcgaagagattgcccaagccaaaggaaaatcaactgctcagatatcattgagatgggtgtacgaacaaggtgtgagcatagtaacaaaaagttacaacaaagaaagaatgaggcagaaccttgacatcttcgacttctgcttgaccgaggaggaactggagaagatgagtcatcttccacagcggaaaggggttacctttgcttcaattctaggaccccatgatattgttctggaagttgacgaagaattatgagctgcaagacttggtcatttattattattagatgtttctcccaattttaccgccgggatatactagctgatgtagtcaagtctattagggaaacatgtgatatcgtttttgtattacttatagtcgatgatatacgtaccgtttgcagcatatacaatttgcagcacctttcctttgttctgtagagatcgatgtatggtacgtatcacgtttcggaaacgtagatacatacatacttcttccccttctcgtcaaaaaaaaaaaaaaaaaaaaaaaaaaaaaa

SEQ ID NO: 2 - protein, 319 aa, Strawberry
MAKVPSVTKSSCGDDIQTMPVIGMGTSSYPRADPETAKAAILEAIRAGYRHFDTAAAYGSEKDLGEAIAEALRLQLIKSRDELFITTKLWASFAEKDLVLFSIKASLSNLQVEYIDMYIIHWPFKLGKEVRTMPVERDLVQPLDIKSVWEAMEECKKLGLARGIGVSNFTSSMLEELLSFAEIPPVVNQLEMNPAWQLKKLRDFCKAKGIHVTAYSPLGAARTKWGDDRVLGSDIIEEIAQAKGKSTAQISLRWVYEQGVSIVTKSYNKERMRQNLDIFDFCLTEEELEKMSHLPQRKGVTFASILGPHDIVLEVDEEL

SEQ ID NO: 3 - DNA, 29 bp, Artificial sequence
  Initiator oligonucleotide AKR1
actgcagtctagacatggcaaaggttcct

SEQ ID NO: 4 - DNA, 22 bp, Artificial sequence
  Initiator oligonucleotide sequence AKR2
aaagctttcataattcttcgtc

(19) United States
(12) Patent Application Publication
(10) Pub. No.: US 2005/0150001 A1
(43) Pub. Date: Jul. 7, 2005

(54) Dna construct and method for increasing production of vitamin C in a plant

(76) Inventors: Maria Fornanda Agius Guadalupe, Malaga (UY); Miguel Angel Botella Mesa, Melaga (ES); Victoriano Valpuesta Fernandez, Melaga (ES)

Correspondence Address: DANN, DORFMAN, HERRELL & SKILLMAN, 1601 MARKET STREET, SUITE 2400, PHILADELPHIA, PA, 19103-2307, US

(21) Appl. No.: 10/499,842

(22) PCT Filed: Dec. 19, 2002
(86) PCT No.: PCT/GB02/05818

(30) Foreign Application Priority Data
Dec. 27, 2001 (ES) P200102896

(51) Int. Cl. A01H 1/00; C12N 15/82; C12P 17/04; C12P 7/58; C12N 9/02

(52) U.S. Cl. 800/278; 435/189; 435/126; 435/137; 435/468

(57) ABSTRACT

Provided are DNA constructs that comprise a DNA molecule encoding a protein with D-galacturonate reductase activity involved in L-ascorbic acid synthesis in plant cells and a region for initiating functional transcription in plants. Preferred constructs include SEQ ID NO: 1 or analogues thereof. The constructs have utility in increasing vitamin C production in plants, and making plants more resistant to stress. Also provided are related materials and methods for performing the invention.

### FIELD OF THE INVENTION

The invention describes a DNA construct that comprises a DNA sequence encoding a protein involved in the synthesis of vitamin C in plant cells and the use of this DNA construct to increase the vitamin C content of plants.

### BACKGROUND OF THE INVENTION

One of the aims of genetic engineering is to obtain plants with features that represent an improvement on nature, whether the plants are wild species or the consequences of agricultural improvements introduced by human beings. As far as fruit-producing plants are concerned, any properties able to increase the nutritional capacity of plants are of the greatest interest. Vitamins are components that improve the nutritional capacity of fruits because these compounds perform an essential role in human metabolism and must be taken in the diet because humans do not have the ability to synthesise them.

In order to manipulate and control a given plant feature in permanent manner, it is necessary to identify and isolate the gene or genes that encodes/encode the protein whose activity modifies the required characteristic. The introduction of a coded sequence of this gene under the control of an appropriate promoter modifies the enzymatic activities of a transgenic plant and this is reflected by a corresponding change in the composition of the transformed plant tissue.

Vitamin C (L-ascorbic acid) is essential to human beings, who are incapable of synthesising or storing it in sufficient quantities in the body. The diet must therefore provide a regular and adequate supply of this vitamin. Vitamin C from plants is the main source of vitamin C in the human diet.

We have long been aware from studies on vitamin C biosynthesis in plants that the biosynthetic pathway in plants is different to that in animals. A vitamin C biosynthesis pathway specific to plants was recently described (Wheeler et al, Nature 393:365-369,1998). This pathway, defined as a non-inversion pathway by Wheeler-Smirnoff, is based on biochemical and genetic evidence. Although this pathway is the most common one in plants, other metabolic pathways that produce ascorbic acid may also exist in plants (Smirnoff et al, Annu. rev. Plant Physiol. Plant Molec. Biol. 52:437-467, 2001). Radio labelling studies have suggested that D-galacturonic acid and also the methyl ester of D-galacturonic acid are both directly transformed to L-ascorbic acid in vivo via a pathway that involves a configuration inversion (Davey et al, J. Sci. Food Agric. 80:825-860, 2000). This specific transformation was discussed many years ago in strawberry fruits (Finkle et al., Biochim. Biophys. Acta 38:332-339, 1960). The methyl ester of galacturonic acid may be reduced by a non-specific aldo-keto reductase that generates L-galactone-1,4-lactone, which is the substrate of the enzyme L-galactone-1,4-lactone dehydrogenase (EC 1.3.2.3), i.e. the last step in L-ascorbic acid synthesis. It has recently shown that this pathway for the production of L-ascorbic acid from the methyl ester of galacturonic acid also occurs in the cells of Arabidopsis plants (Davey et al., Plant Physiol. 121:535-543, 1999).

Although genes responsible for synthesising the proteins that catalyse some of the steps in the various metabolic pathways that lead to the biosynthesis of L-ascorbic acid in plants have been identified and isolated, some of the genes that encode proteins involved in vitamin C synthesis have not been identified or characterised. These include the gene that encodes a protein with aldo-keto reductase activity responsible for catalysing the reduction of D-galacturonic acid to L-galactonic acid. This could explain the existence of ascorbic acid synthesis from the methyl ester of D-galacturonic acid in certain plant tissues (Davey et al., J. Sci. food Agric. 80:825-860, 2000). Identification and characterisation of this gene would allow us to obtain plants with a higher vitamin C content.

Because a need exists to produce plants with an increased vitamin C content, we also therefore need to modify plants to increase their in vivo vitamin C production and thus also their nutritional value.

### BRIEF DESCRIPTION OF THE INVENTION

In general terms, the invention tackles the problem of increasing the vitamin C content of plants or plant products such as fruit, particularly plants and fruit consumable by human beings unable to synthesis vitamin C, thus increasing the nutritional value of these plants and fruits.

The solution provided by this invention is based in the identification, isolation and characterisation of a DNA molecule that encodes a protein involved in vitamin C synthesis in plants. More specifically, the invention concerns a protein that acts to increase vitamin C synthesis in plant cells in which it is active. The effectiveness of the activity encoded by this DNA molecule has been demonstrated in transgenic Arabidopsis plant. Because these plants product the protein encoded by this DNA molecule, the vitamin C content of these plants compared to untransformed wild type plants is considerably and significantly increased. Conversely, studies on a series of species from the genus Fragaria have revealed a strong positive correlation between the presence of the protein encoded by the FaGalUR gene, more specifically a protein with D-galacturonate reductase activity, and L-ascorbic content.

One object of this invention is consequently to provide a DNA construct that comprises a DNA molecule that encodes a protein involved in the synthesis of vitamin C in plants and a promoter DNA sequence that regulates the expression of this coded DNA molecule.

Another aspect of this invention is to use the said encoding DNA molecule or the said DNA construct to increase vitamin C production in a plant or a plant product.

Another aspect of this invention is to provide a vector to contain this coded DNA molecule or DNA construct. Cells transformed using this vector constitute an additional object of this invention.

Another aspect of this invention is to use the said coding DNA molecule or the said DNA construct in the production of transgenic plants that specifically express the coded protein. The resulting transgenic plants that provide an increased vitamin C content constitute another additional object of this invention.

Another aspect of this invention is to provide a protein with aldo-keto reductase activity that reduces D-galacturonic acid or a protein with D-galacturonate reductase activity, obtained by the expression and translation of the said DNA molecule.

### BRIEF DESCRIPTION OF THE DRAWINGS

FIG. 1 shows a schematic representation of the binary vector that is used to transform Arabidopsis thaliana plants.

FIG. 2 shows ascorbic acid content correlates with the expression of GalUR. (A) Photographs of flowers and fruits at the sampled developmental stages: F1, young flower; F2, mature flower; F3, small green fruit ; F4, green fruit; F5, white fruit; F6, intermediate fruit; F7, red fruit. (B) Northern blot of total RNA extracted from strawberry fruits, at different ripening stages (F1-F7), and leaves (L). (C) Ribosomal rRNA is shown as a loading control. (D) GalUR protein content, determined by immunoblot, at different ripening stages F1 to F7. (E) Ascorbic acid content of the tissues described in (A), (F) GalUR protein content, determined by immunoblot, corresponding to red-ripe fruits of Fragaria x ananassa (cv. Chandler) (1), Fragaria chiloensis (2), Fragaria virginiana (3) and Fragaria moschata (4). (G) Ascorbic acid content of the same Fragaria species described in F.

FIG. 3 shows overexpression of GalUR in Arabidopsis thaliana increases the ascorbic acid content. (A) Immunoblot of extracts prepared from young seedlings of 17 independent Arabidopsis transgenic lines transformed with the sense construct of the GalUR gene under the control of the constitutive promoter 35SCaMV. The immunoblot was performed as described in the Methods except that 10 μg of total protein was used. (B) Ascorbic acid content in the extracts of control (pSOV2) and three homozygous Arabidopsis thaliana transgenic lines, numbers 3, 10 and 17. Ascorbic acid was determined by the ascorbate oxidase assay (Rao & Ormrod (1995) 61, 71-78). Total ascorbic acid content was determined by measuring the absorbance at 265 nm after addition of 4 U of ascorbate oxidase (Sigma) to the reaction medium containing the plant extract and 100 mM potassium phosphate pH 5.6. Plant extracts were obtained from liquid nitrogen frozen tissue, macerated in 2 mM metaphosphoric acid containing 2% (w/v) EDTA. Samples were compared to a standard curve with known concentrations of ascorbic acid.

### DETAILED DESCRIPTION OF THE INVENTION

The invention refers to a DNA construct, hereafter referred to as the DNA construct of the invention, that comprises a DNA molecule encoding a protein with D-galacturonate reductase activity (FaGalUR EC 1.1.1.19) involved in L-ascorbic acid synthesis in plant cells and a region responsible for initiating functional transcription in plants.

Nucleic acid according to the present invention may include cDNA, RNA, genomic DNA and modified nucleic acids or nucleic acid analogs (e.g. peptide nucleic acid). Where a DNA sequence is specified, e.g. with reference to a figure, unless context requires otherwise the RNA equivalent, with U substituted for T where it occurs, is encompassed. Nucleic acid molecules according to the present invention may be provided isolated and/or purified from their natural environment, in substantially pure or homogeneous form, or free or substantially free of other nucleic acids of the species of origin. Where used herein, the term “isolated” encompasses all of these possibilities. The nucleic acid molecules may be wholly or partially synthetic. In particular they may be recombinant in that nucleic acid sequences which are not found together in nature (do not run contiguously) have been ligated or otherwise combined artificially. Alternatively they may have been synthesised directly e.g. using an automated synthesiser.

The term “plant” as used in this description includes the plant per se and also its parts, for example, leaves, stems, roots and propagation material and also seeds, flowers, fruits and tubers. The DNA molecule that encodes the protein with D-galacturonate reductase activity involved in L-ascorbic acid synthesis in plant cells, hereafter referred to as DNA molecule of the invention, is a DNA molecule selected from among:
a) a DNA molecule that includes the nucleotides sequence shown in SEQ, ID. No.: 1; and
b) a DNA molecule similar to the sequence defined in a) that
i) is substantially homologous to the DNA sequence defined under a); and/or
ii) encodes a polypeptide that is substantially homologous to the protein encoded by the DNA sequence defined under a).

The DNA molecule of the invention encodes a protein with D-galacturonate reductase activity involved in L-ascorbic acid synthesis in plant cells, preferably a D-galacturonate reductase obtainable from strawberry. This protein with D-galacturonate reductase activity is involved in vitamin C synthesis because it catalyses the reduction of D-galacturonic acid to L-galactonic acid. This allows vitamin C synthesis to be expressed from galacturonic acid or the methyl ester of D-galacturonic acid in certain plant tissues (Davey et at., J. Sci. Food Agric. 80:825-860, 2000). The use of this DNA molecule or DNA construct of the invention would therefore allow plants with an increased vitamin C content to be obtained.

In the sense used in the description, the term “analogue” includes any DNA molecule that displays the same functions as the DNA molecule defined under a), i.e. that encodes a protein with D-galacturonate reductase activity involved in L-ascorbic acid synthesis in plant cells. This analogue DNA molecule could contain conservative or non-conservative nucleotide substitutions (e.g. encoding conservative variation within the polypeptide, i.e. substitution of one hydrophobic residue such as isoleucine, valine, leucine or methionine for another, or the substitution of one polar residue for another, such as arginine for lysine, glutamic for aspartic acid, or glutamine for asparagine) or could contain one or more additional nucleotides at any of its endings, or could contain one or more deletions.

In general, the analogue DNA molecule is substantially homologous to the nucleotide sequence identified as SEQ. ID. No: 1. In the sense used in this description, the expression “substantially homologous”, as applied to nucleotides sequences, means that the nucleotide sequences in question display a homology or degree of identity to of at least 60% or preferably at least 80% or most preferably at least 95%.

Homology or identity may be as defined and determined by the TBLASTN program, of Altschul et al. (1990) J. Mol. Biol. 215: 403-10, or BestFit, which is part of the Wisconsin Package, Version 8, September 1994, (Genetics Computer Group, 575 Science Drive, Madison, Wis., USA, Wisconsin 53711). Preferably sequence comparisons are made using FASTA and FASTP (see Pearson & Lipman, 1988. Methods in Enzymology 183: 63-98). Parameters are preferably set, using the default matrix, as follows:
Gapopen (penalty for the first residue in a gap): −12 for proteins/−16 for DNA
Gapext (penalty for additional residues in a gap): −2 for proteins/−4 for DNA
KTUP word length: 2 for proteins/6 for DNA.

The DNA molecule in the invention may be obtained from any organism that contains the molecule in its natural form, for example, the strawberry (Fragaria x ananassa) or a host organism transformed using this DNA molecule. Alternatively, the DNA molecule of the invention may be isolated using conventional methods from the DNA of any organism by the use of probes and oligonucleotides prepared using information on the sequence of the DNA molecule of the invention provided in this description.

In one specific embodiment, the DNA molecule of the invention comprises or consists of the nucleotide sequence displayed in SEQ. ID. No. 1. The nucleotide sequence shown in SEQ. ID. No. 1 is a known sequence filed by the research group to which the inventors belong in GeneBank under access number AF039182. The description states that this DNA sequence encodes a protein with chalcone reductase activity but does not, however, consider the possibility that this protein is involved in the in vivo synthesis of vitamin C in plant cells. Now it has been possible to determine that the nucleotide sequence shown in SEQ. ID. No. 1 corresponds to a DNA copy (cDNA) of the FaGalUR gene from the strawberry (Fragaria x ananassa) encoded by a D-galacturonate reductase protein (FaGalUR) that is involved in vitamin C synthesis in plant cells because it catalyses the reduction of D-galacturonic acid to L-galactonic acid.

In another embodiment, the DNA molecule of the invention comprises or consists of the nucleotide sequence displayed in SEQ. ID. No. 1. but wherein the C in positions 660 and 757 is substituted for a T.

The DNA molecule of the invention may be obtained using conventional methods, eg. methods for isolating and identifying nucleic acids from any organism that contains the molecule or from a host organism transformed using this DNA molecule.

Alternatively, the DNA molecule of the invention may be isolated using conventional methods from the DNA of any other organism by the use of probes and oligonucleotides prepared using the information on the DNA sequence provided by this invention. Thus one aspect of the invention may include:
(a) providing a preparation of nucleic acid, e.g. from plant cells. Test nucleic acid may be provided from a cell as genomic DNA, cDNA or RNA, or a mixture of any of these, preferably as a library in a suitable vector. If genomic DNA is used the probe may be used to identify untranscribed regions of the gene (e.g. promoters etc.), such as are described hereinafter,
(b) providing a nucleic acid molecule which is a probe or primer based on SEQ ID. No. 1,
(c) contacting nucleic acid in said preparation with said nucleic acid molecule under conditions for hybridisation of said nucleic acid molecule to any said gene or homologue in said preparation, and,
(d) identifying said gene or homologue if present by its hybridisation with said nucleic acid molecule. Binding of a probe to target nucleic acid (e.g. DNA) may be measured using any of a variety of techniques at the disposal of those skilled in the art. For instance, probes may be radioactively, fluorescently or enzymatically labelled. Other methods not employing labelling of probe include amplification using PCR (see below), RN'ase cleavage and allele specific oligonucleotide probing. The identification of successful hybridisation is followed by isolation of the nucleic acid which has hybridised, which may involve one or more steps of PCR or amplification of a vector in a suitable host

In a further embodiment, hybridisation of nucleic acid molecule to a variant may be determined or identified indirectly, e.g. using a nucleic acid amplification reaction, particularly the polymerase chain reaction (PCR). PCR requires the use of two primers to specifically amplify target nucleic acid, so preferably two nucleic acid molecules with sequences characteristic of VRN1 are employed. Using RACE PCR, only one such primer may be needed (see “PCR protocols; A Guide to Methods and Applications”, Eds. Innis et al, Academic Press, New York, (1990)). Example 1 describes a method for obtaining cDNA containing the full sequence of nucleotides encoding strawberry protein FaGalUR and sequence amplification by PCR using the primers of SEQ ID. Nos 3 and 4.

Thus a method involving use of PCR in obtaining nucleic acid according to the present invention may include:
(a) providing a preparation of plant nucleic acid, e.g. from a seed or other appropriate tissue or organ,
(b) providing a pair of nucleic acid molecule primers useful in (i.e. suitable for) PCR e.g. based on SEQ ID No 1, 3 or 4,
(c) contacting nucleic acid in said preparation with said primers under conditions for performance of PCR,
(d) performing PCR and determining the presence or absence of an amplified PCR product. The presence of an amplified PCR product may indicate identification of a variant.

The DNA construct of the invention comprises, in addition to the DNA molecule of the invention, a region for initiating functional transcription in plants, ie. a promoter DNA sequence that regulates the expression of the encoding DNA molecule of the invention. In this DNA construct of the invention, any of the endings (3′ or 5′) of the DNA molecule of the invention, may be joined to the 3′ ending of the transcription initiator region. The DNA construct of the invention may also contain an operably linked transcription termination sequence. In one specific realisation, the region initiating functional transcription in plants is the CaMV 35S promoter. Other examples are disclosed at pg 120 of Lindsey & Jones (1989) “Plant Biotechnology in Agriculture” Pub. OU Press, Milton Keynes, UK. The promoter may be selected to include one or more sequence motifs or elements conferring developmental and/or tissue-specific regulatory control of expression. Inducible plant promoters include the ethanol induced promoter of Caddick et al (1998) Nature Biotechnology 16: 177-180.

In one embodiment, the promoter is an inducible promoter. The term “inducible” as applied to a promoter is well understood by those skilled in the art. In essence, expression under the control of an inducible promoter is “switched on” or increased in response to an applied stimulus. The nature of the stimulus varies between promoters. Some inducible promoters cause little or undetectable levels of expression (or no expression) in the absence of the appropriate stimulus. Other inducible promoters cause detectable constitutive expression in the absence of the stimulus. Whatever the level of expression is in the absence of the stimulus, expression from any inducible promoter is increased in the presence of the correct stimulus.

The DNA molecule of the invention or the DNA construct of the invention may be inserted into an appropriate vector. The invention therefore also refers to a vector, such as an expression vector, that comprises the DNA molecule or construct of the invention. The choice of vector depends on the host cell into which the vector will later be introduced. As an example, the vector into which this DNA sequence is introduced may be a plasmid or a vector that becomes part of the genome of the cell and replicates together with the chromosome (or chromosomes) of which it becomes part when it is introduced into a host cell.

In the vector provided by this invention, the DNA molecule of the invention is operably linked to a promoter and a terminal sequence. The promoter may be any DNA sequence that displays transcriptional activity in the selected host cell and may be derived from genes that encode homologous or heterologous host cell proteins. The procedures used to bond the DNA sequence of the invention to the promoter and the terminal sequence, respectively, and to insert this construction into a vector are well known to experts in the field and have been described, for example, by Sambrook et al., (1989). Molecular Cloning. A Laboratory Manual. Cold Spring Harbor, N.Y.

Particularly of interest in the present context are nucleic acid constructs which operate as plant vectors. Specific procedures and vectors previously used with wide success upon plants are described by Guerineau and Mullineaux (1993) (Plant transformation and expression vectors. In: Plant Molecular Biology Labfax (Croy RRD ed) Oxford, BIOS Scientific Publishers, pp 121-148). Suitable vectors may include plant viral-derived vectors (see e.g. EP-A-194809).

The invention also provides a cell, for example, a plant cell, that includes a DNA sequence of the invention, or a DNA construct of the invention or the vector mentioned above. Host cells that may be transformed the DNA molecule of the invention or the DNA construct of the invention may be prokaryotic cells or preferably, eukaryotic cells such as plant cells.

In the cells and plants of the present invention, the construct or vector is heterologous. The term “heterologous” is used broadly in this aspect to indicate that the gene/sequence of nucleotides in question have been introduced into said cells of the plant or an ancestor thereof, using genetic engineering, i.e. by human intervention. A heterologous gene may replace an endogenous equivalent gene, i.e. one which normally performs the same or a similar function, or the inserted sequence may be additional to the endogenous gene or other sequence. Nucleic acid heterologous to a plant cell may be non-naturally occurring in cells of that type, variety or species. Thus the heterologous nucleic acid may comprise a coding sequence of or derived from a particular type of plant cell or species or variety of plant, placed within the context of a plant cell of a different type or species or variety of plant. A further possibility is for a nucleic acid sequence to be placed within a cell in which it or a homologue is found naturally, but wherein the nucleic acid sequence is linked and/or adjacent to nucleic acid which does not occur naturally within the cell, or cells of that type or species or variety of plant, such as operably linked to one or more regulatory sequences, such as a promoter sequence, for control of expression.

Plant cells may be transformed using conventional methods. For a review of gene transference to plants, including vectors, DNA transference methods etc, see, for example, the book entitled “Ingenieria génetica y transferencia génica”, by Marta Izquierdo, Ed. Pirámide (1999), in particular chapter 9 entitled “Transferencia génica a plantas”, pages 283-316.

Nucleic acid can be introduced into plant cells using any suitable technology, such as a disarmed Ti-plasmid vector carried by Agrobacterium exploiting its natural gene transfer ability (EP-A-270355, EP-A-0116718, NAR 12(22) 8711-87215 1984), particle or microprojectile bombardment (U.S. Pat. No. 5,100,792, EP-A-444882, EP-A-434616) microinjection (WO 92/09696, WO 94/00583, EP 331083, EP 175966, Green et al. (1987) Plant Tissue and Cell Culture, Academic Press), electroporation (EP 290395, WO 8706614 Gelvin Debeyser) other forms of direct DNA uptake (DE 4005152, WO 9012096, U.S. Pat. No. 4,684,611), liposome mediated DNA uptake (e.g. Freeman et al. Plant Cell Physiol. 29: 1353 (1984)), or the vortexing method (e.g. Kindle, PNAS U.S.A. 87: 1228 (1990d) Physical methods for the transformation of plant cells are reviewed in Oard, 1991, Biotech. Adv. 9: 1-11.

The invention also provides a protein with D-galacturonate reductase activity involved in the in vivo synthesis of L-ascorbic acid in plant cells, and uses thereof, hereafter referred to as protein of the invention, which contains an aminoacid sequence selective from among:
a) an amino acid sequence that includes the amino acid sequence demonstrated in SEQ. ID. No. 2 and
b) a substantially homologous amino acid sequence that is functionally equivalent to the amino acid sequences defined under a).

In the sense used in this description, the expression “substantially homologous” means that the aminoacid sequences in question display a degree of identity, at aminoacid level, of at least 60%, preferably at least 80% and most preferably at least 95%.

Similarly, in the sense used in this description, the expression “functionally equivalent” means that the protein in question is a D-galacturonate reductase involved in the production of L-ascorbic acid in plant cells due to its action in catalysing the reduction of D-galacturonic acid to L-galactonic acid. D-galacturonate reductase activity may be determined by means of a procedure such as the one described in example 1.5.

In one embodiment, the protein of the invention contains an amino acid sequence that comprises or consists of the amino acid sequence demonstrated in SEQ. ID. No. 2. SEQ. ID. No. 2 corresponds to the amino acid sequence deduced from the nucleotide sequence in SEQ. ID. No. 1 and includes the expression product of the strawberry gene FaGalUR, specifically D-galacturonate reductase FaGalUR. This protein has a theoretical molecular weight of 35.7 kDa and an isoelectric point (pI) of 5.3. D-galacturonic acid is a substrate of this immunopurified enzyme.

In another embodiment, the protein of the invention contains an amino acid sequence that comprises or consists of the amino acid sequence demonstrated in SEQ. ID. No. 2 but wherein the alanine at position 186 is a valine.

The protein of the invention may be obtained by means of a method that involves culturing an appropriate host cell containing the DNA molecule of the invention or DNA construct of the invention under conditions that allow protein production and recovery from the culture medium. Alternatively, the protein of the invention may be obtained from a producing organism by means of a procedure that includes cultivation of the producing organism, for example strawberry fruit, under conditions appropriate for the expression of the said protein and then recovery of the protein.

The invention also refers to the use of the DNA molecule of the invention or DNA construct of the invention in the production of transgenic plants that specifically express the protein encoded by the DNA molecule of the invention and provide an increased vitamin C content.

The DNA molecule of the invention or the DNA construct of the invention may be used in processes for the improvement of plants consumable by human beings unable to synthesis vitamin C: for example, plants consumable by human beings or by animals or plants that produce fruit consumable by human beings or by animals. These transgenic plants may be obtained by the conventional methods described previously and provide a higher nutritional value as a result of their increased vitamin C content.

The invention therefore provides a transgenic cell, such as a transgenic plant cell, that comprises a DNA construct of the invention.

A transgenic plant that comprises at least one of the said transgenic plant cells constitutes an additional object of this invention. In one specific realisation, the said transgenic plant is a plant consumable by human beings incapable of synthesising vitamin C, for example, a plant consumable by human beings or by animals or a plant producing fruit consumable by human beings or by animals for example, strawberry, tomato, maize, wheat, rice, potato, lettuce, etc.

The invention also refers to a method for increasing vitamin C production in a plant producing vitamin C in vivo that includes introducing the DNA construct of the invention into a plant that contains a promoter functional in the said plant that is operably linked to the DNA molecule of the invention.

For the purposes of illustration, this plant may be any plant that produces vitamin C in vivo, preferably any plant that produces in vivo vitamin C consumable by human beings incapable of synthesising vitamin C, for example, a plant consumable by human beings or by animals or a plant producing fruit consumable by human beings or by animals, for example, strawberry, tomato, maize, wheat, rice, potato, lettuce, etc. In one specific example, the invention provides a method for increasing the production of L-ascorbic acid in strawberry fruits that includes introducing a DNA construct of the invention into a strawberry plant.

The use of the DNA construct of the invention comprising a DNA molecule responsible for encoding a protein involved in vitamin C synthesis in plant cells involves increasing the flow through the metabolic pathway that leads to vitamin C synthesis from D-galacturonic acid or the methyl ester of D-galacturonic acid in those plant tissues where the precursor is present. The fact that phenotypic differences cannot be observed between transformed and non-transformed A. thaliana (see example 1.1) allows us to deduce that no deleterious effect arises in transgenic plants that overexpress the said DNA molecule of the invention. Furthermore, to the extent that the vitamin C content of plants is an indicator of the stored reduction potential of plants, the high levels in transgenic plants could suggest that they are advantageously equipped to combat the oxidative stress to which they may be subject during growth and development.

Indeed, results (not shown) confirm that plants of the present invention, i.e. expressing heterologous GalUR, show increased resistance to stress, particularly oxidative stress.

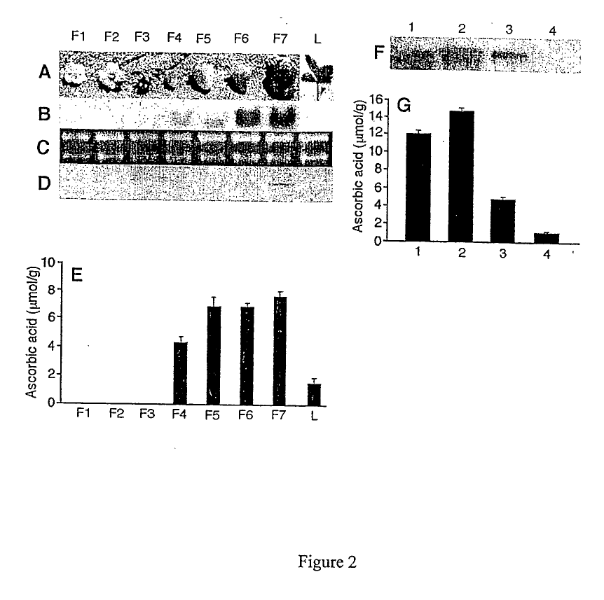

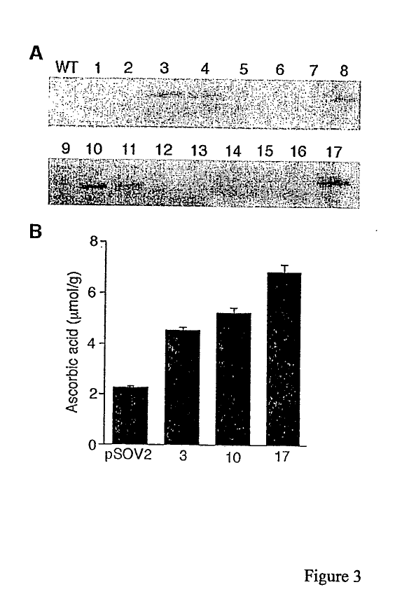

Conversely, a study of the expression of the FaGalUR gene at different strawberry fruit ripening stages has demonstrated the existence of a strong positive correlation between FaGalUR gene expression, the FaGalUR protein and L-ascorbic acid content throughout different strawberry fruit ripening stages (see FIG. 2). This allows us to deduce that levels of mRNA, FaGalUR protein and L-ascorbic acid content are correlated at different strawberry fruit ripening stages during the ripening process. Similarly, a study of the expression of the FaGalUR gene in various species of the Fragaria genus has shown a strong positive correlation between the presence of the protein coded by the gene FaGalUR and L-ascorbic acid content (see FIG. 3.).

The use of a DNA construct of the invention, particularly a DNA construct that contains a DNA molecule encoding strawberry protein FaGalUR could give rise to transgenic plants that provide fruits with a higher nutritional value due to their increased vitamin C content without any loss of remaining organoleptic properties. This would be reflected in a higher financial value.

The following examples illustrate this invention and should not be considered limiting to the scope of the invention.

### EXAMPLE 1

### Obtaining Transgenic Plants That Express the FaGalUR Gene Product

1.1 Obtaining Transgenic Plants that Express the FaGalUR Gene Product

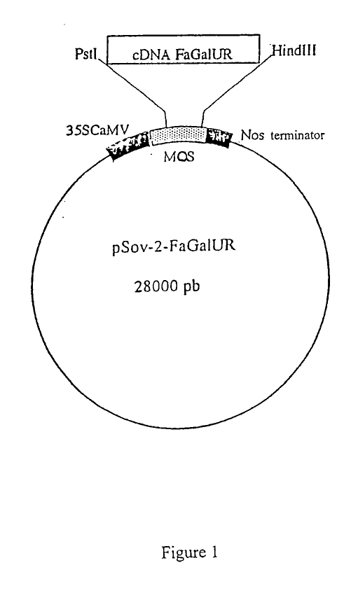

cDNA of strawberry fruits (Fragaria xananassa) containing a sequence encoding FaGalUR protein was isolated by screening using a specific probe for 750 base pairs (pb) for the 5′ end region of the FaGalUR gene using a strawberry fruit cDNA gene library and the specific primers AKR1 and AKR2
AKR1: ACTGCAGTCTAGACATGGCAAAGGTTCCT [SEQ. ID. NO. 3]; and
AKR2: AAAGCTTTCATAATTCTTCGTC [SEQ. ID. NO. 4]
   that contain recognition sites for the endonucleases Pstl and HindIII respectively in their 5′ endings. The amplified product consisting of some 950 pb was cloned in the vector pBSKII previously digested using restriction enzyme EcoRV. The intermediate construct pBSKII-FaGalUR was digested using restriction enzymes Pstl and HindIII and the DNA fragment remaining after digestion that represents the entire open reading frame of the FaGalUR gene was cloned at the multiple cloning site of binary vector pSOV2 (Myle & Botella, 1998 Plant. Mol. Biol. Rep. 00: 1-6) that uses as a transgenic plant selection system the phosphinothricin herbicide resistance gene (ppt) (Thompson et al, 1987 EMBO J. 6: 2519-2524) marketed under the name of BASTA, under the control of CaMV 35S constitutive promoter. The pSOV2-FaGalUR construct (FIG. 1) was transferred by triparental conjugation to the Agrobacterium tumefaciens strain GV3101 to transform Arabidopsis thaliana plants by in planta infiltration with GV3101-FaGalUR A. tumefaciens cultures.

The transgenic plants were analysed using two methods: (i) herbicide resistance test ppt (BASTA) and (ii) by transference and immunodetection of proteins using anti-FaGalUR serum. Most of the plants obtained by transforming with CDNA of the FaGalUR gene display an apparently normal phenotype when compared with control plants transformed using the vector pSOV2 and untransformed plants.

1.2 Northern Blot Analysis

Northern Blot analysis for the purpose of identifying mRNA corresponding to the FaGalUR gene was carried out by conventional means (Sambrook et al 1989, Molecular Cloning: A Laboratory Manual. Ed Cold Spring Harbor Laboratory). FIG. 2A shows the expression of the FaGalUR gene at the various strawberry fruit ripening stages.

1.3 Western Blot Analysis

Western Blot analysis for the purpose of detecting the FaGalUR protein was carried out using conventional methods (Harlow and Lane, 1988. Antibodies: A Laboratory Manual. Ed Cold Spring Harbor Laboratory). FIG. 2B shows the detection of the FaGalUR protein encoded by the gene FaGalUR throughout the various strawberry fruit ripening stages. FIG. 3B, on the other hand, shows the detection of the FaGalUR protein in different Fragaria genus species.

1.4 Quantitative Determination of L-ascorbic Acid

Quantitative determination of L-ascorbic acid (AA) was carried out by following the protocol described by Rao and Ormrod (Rao & Ormrod 1995. Photochem. Photobiol 61:71-78). According to this method, a metaphosphoric acid solution (2% metaphosphoric acid, EDTA 2 mM) was added to 100 mg of A. Thaliana plant macerated with liquid nitrogen, homogenised and then centrifuged at 17,000 rpm for 10 minutes. The resulting supernatant was neutralised at pH 5.6 by adding a 10% sodium citrate solution. After neutralisation, the AA content of extracts of wild type (WT) plants and two transgenic lines referred to as 10 and 17 was determined by measuring the decrease in absorption at 265 mm after adding the enzyme ascorbate oxidase (4 U) to a reaction mixture that contained 100 ml mM pH 5.6 phosphate buffer and 100 ml plant extract made up to a final volume of 1 ml.

Total AA content was determined for the entire plant and the AA content of WT plants was taken as a standard value, which agreed with values obtained by other authors (Rao & Ormrod, 1995, see above).

The AA content results obtained are shown in the following figures: FIG. 2C shows total AA content for the different strawberry fruit ripening stages; FIG. 3A shows total AA content in different species of the Fragaria genus—and FIG. 4B shows the AA content of transgenic plants 10 and 17 that express the product of the FaGalUR gene and in WT plants, quantitatively determined by the spectrophotometric measurement of plants grown under normal conditions.

1.5 Determination of D-galacturonate Reductase Activity

D-galacturonate reductase activity in raw extracts of transformed and untransformed A. thaliana plants was measured using the procedure described below. Briefly, 1 g of tissue macerated in liquid nitrogen was homogenised in 2 ml of extraction buffer [50 mM pH 7.2 sodium phosphate buffer, 2 mM EDTA-Na₂, 2 mM DTT (dithiothreitol), 0.1 g/g tissue PNPP (polyvinylpolypyrrolidone) and 20% glycerol (v/v)] and a Turrax homogeniser. The homogenised tissue was then filtered through a Miracloth paper and centrifuged at 6000 g for 30 minutes at 4° C. The supernatant was then transferred to a new tube. Protein concentration was quantified using the Bradford method (BIO-RAD).

The activity was tested using the same extraction buffer to which was added a final concentration of 30 mM of the substrates used, 100 μl of the extract prepared from WT Arabidopsis or of the transgenic plants overexpressing FaGalUR (10 and 17) and 1 mM of NADPH+H⁺ or NAD+. The final volume of each test was 1 ml and the reaction was monitored by measuring the change in absorbance at 340 nm using a Shimatzu 1600 spectrophotometer. The substrates used in each reaction were as follows: L-galactose, D-glucuronic and D-galacturonic. The results obtained are shown in table 1. This shows the enzyme activity of the FaGalUR protein. 1 enzyme unit (U) is defined as the amount of enzyme able to decrease absorbance by 0.001 at 340 nm for 1 minute. Activity is expressed as U/mg protein.

TABLE 1
Enzymatic activity of the FaGalUR protein in various
extracts using different substrates

| | D-galacturonic | | |
|---|---|---|---|
| Extract | acid | D-glucuronic Acid | L-galactose |
| WT | 0 | 0 | 0 |
| 10 | 28 ± 4 | 0 | 0 |
| 17 | 72 ± 6 | 0 | 0 |

Enzymatic activity studies were then carried out using FaGalUR protein immunopurified from transgenic Arabidopsis plants that overexpress the FaGalUR gene (more specifically, those identified as 10 and 17). The protocol used was the same used for Arabidopsis plant extracts. The first results obtained with immunopurified enzyme show that the FaGalUR protein is able to reduce the D-galacturonic acid substrate and displays an activity of 220 U/mg protein.

1.6 Discussion

The vitamin C (L-ascorbic acid) content of the two independent transgenic A. thaliana lines that overexpress the FaGalUR strawberry gene (proven by means of Northern Blot and Western Blot analysis) was significantly higher than that of non-transgenic plants. Because studies using labelled precursors have proved that the vitamin C biosynthesis pathway from D-galacturonic acid (and from its methyl ester) is operational in Arabidopsis, overexpression of FaGalUR in transgenic A. thaliana plants would have the effect of increasing vitamin C content by reinforcing this metabolic pathway. This has also been shown to be the main metabolic pathway for the biosynthesis of vitamin C (from L-galactose) In Arabidopsis. Activation of the pathway with D-galacturonic acid as its starting point by the introduction of FaGalUR allows very significant increases to be obtained in vitamin C content.

Conversely, when D-galacturonate reductase activity (NADP-dependent) is measured in raw extract of transformed and non-transformed A. thaliana plants, it has been proven that measured enzymatic activity is significantly higher in transgenic A. thaliana plants. Moreover, the immunopurification to electrophoretic homogeneity of GalUR protein expressed in Arabidopsis, demostrate the specificity of this enzyme by the substrate D-galacturonic acid. This is additional evidence of the reducing action of D-galacturonic acid on the product of strawberry FaGalUR gene expression. This would explain the higher vitamin C content of transgenic plants that only differ from non-transgenic plants in possessing a copy of the FaGalUR gene in their genome.

1. A DNA construct that comprises a DNA molecule encoding a protein with D-galacturonate reductase activity involved in L-ascorbic acid synthesis in plant cells and a region for initiating functional transcription in plants.

2. The DNA construct of claim 1, wherein said DNA molecule encoding a protein with D-galacturonate reductase activity involved in L-ascorbic acid synthesis in plant cells is a DNA molecule selected from the following:
a) a DNA molecule that includes the nucleotide sequence shown in SEQ ID NO: 1; and
b) a DNA molecule analogous to the sequence defined under a) that
(i) is substantially homologous to the DNA sequence defined under a) and
(ii) encodes a polypeptide that is substantially homologous to the protein encoded by the DNA sequence defined under a).

3. The DNA construct of claim 2, wherein said DNA molecule that encodes a protein with D-galacturonate reductase activity involved in L-ascorbic acid synthesis in plant cells is a DNA molecule that comprises or consists of the nucleotide sequence shown in SEQ ID NO: 1.

4. The DNA construct of claim 1 further comprising an operably linked transcription termination sequence.

5. A recombinant vector that includes the DNA construct of claim 1.

6. A cell that includes the DNA construct of claim 1.

7. A method of catalysing the reduction of D-galacturonic acid to L-galactonic acid, the method comprising contacting the D-galacturonic acid with a protein that includes an amino acid sequence selected from:
(a) an amino acid sequence that includes the amino acid sequence shown in SEQ ID NO: 2, and
(b) an amino acid sequence substantially homologous and functionally equivalent to the amino acid sequences defined under a).

8. The method of claim 7 wherein the protein comprises or consists of the amino acid sequence shown in SEQ ID NO: 2.

9. The method of claim 7 wherein the protein is provided by culturing a cell that includes a DNA construct comprising a DNA molecule encoding a protein with D-galacturonate reductase activity involved in L-ascorbic acid synthesis in plant cells and a region for initiating functional transcription in plants, under conditions that allow the said protein to be produced and recovered from the culture medium.

10. A transgenic plant cell that comprises the DNA construct of claim 1.

11. A transgenic plant that includes at least one transgenic plant cell of claim 10.

12. The transgenic plant of claim 11, wherein said plant is an in vivo producer of vitamin C consumable by human beings incapable of synthesising vitamin C.

13. The transgenic plant of claim 11, where said plant is an in vivo producer of vitamin C consumable by human beings or animals or a plant producing fruit consumable by human beings or animals.

14. The transgenic plant of claim 13, where said plant is selected from a group made up of strawberries, tomatoes, maize, wheat, rice, potatoes and lettuce.

15. (canceled)

16. A method for transforming a cell which comprises the step of introducing the construct of claim 1 into a host cell, and optionally causing or allowing recombination between said construct and the host cell genome such as to transform the host cell.

17. A method for producing a transgenic plant, which method comprises the steps of:
(a) introducing the construct of claim 1 into a plant cell, and optionally causing or allowing recombination between said construct and the plant cell genome, thereby effecting transformation of said plant cell,
(b) regenerating a plant from the transformed plant cell, said regenerated plant having increased vitamin C content as compared with the non-transgenic plant.

18. A method for increasing vitamin C production in a plant producing vitamin C in vivo, said method comprising introducing the DNA construct of claim 1 into said plant.

19. A method for increasing the resistance to oxidative stress of a plant, said method comprising introducing the DNA construct of claim 1 into the said plant.

20. A method for the isolation of a DNA molecule encoding a protein with D-galacturonate reductase activity, said method comprising:
(a) contacting a preparation of nucleic acids with a probe comprising a nucleic acid sequence which is all or part of SEQ ID NO: 1, under hybridization conditions;
(b) identifying hybridization with said probe; and
(c) isolating the nucleic acid hybridized with said probe, thereby isolating a DNA molecule encoding a protein with D-galacturonate reductase activity.

21. The DNA construct of claim 1, wherein said DNA molecule encoding a protein with D-galacturonate reductase activity involved in L-ascorbic acid synthesis in plant cells is a DNA molecule selected from the following:
a) a DNA molecule that includes the nucleotide sequence shown in SEQ ID NO: 1; and
b) a DNA molecule analogous to the sequence defined under a) that
(i) is substantially homologous to the DNA sequence defined under a), or
(ii) encodes a polypeptide that is substantially homologous to the protein encoded by the DNA sequence defined under a).

22. A recombinant vector that includes the DNA construct of claim 2.

23. A cell that includes the DNA construct of claim 2.

24. A cell that includes the recombinant vector of claim 5.

25. A cell that includes a the recombinant vector of claim 22.

26. The method of claim 7 wherein the protein is provided by culturing a cell that includes a recombinant vector comprising a DNA molecule encoding a protein with D-galacturonate reductase activity involved in L-ascorbic acid synthesis in plant cells and a region for initiating functional transcription in plants, under conditions that allow said protein to be produced and recovered from the culture medium.
